(19) United States
(12) Patent Application Publication
(10) Pub. No.: US 2023/0135376 A1
(43) Pub. Date: May 4, 2023

(54) MULTIPHASIC CONTRACEPTIVE AND/OR HORMONE REPLACEMENT THERAPY

(71) Applicant: Millicent Pharma Limited, Dundalk, County Louth (IE)

(72) Inventor: Roger M. Boissonneault, Branford, CT (US)

(21) Appl. No.: 17/431,470

(22) PCT Filed: Jun. 18, 2021
(86) PCT No.: PCT/IB2021/055408

§ 371 Date: Aug. 17, 2021

Related U.S. Application Data
(60) Provisional application No. 63/044,047, filed on Jun. 25, 2020.

(51) Int. Cl. A61K 31/565; A61K 31/57

(52) U.S. Cl. CPC A61K 31/565; A61K 31/57

(57) ABSTRACT

A multiphasic contraceptive and/or hormone replacement therapy regimen that provides for a low level of estrogen throughout the regimen. Also described is a kit that may be used for the multiphasic contraceptive and/or hormone replacement therapy regimen.

The present application claims the benefit of U.S. Provisional Application No. 63/044,047, filed Jun. 25, 2020, the entirety of which is incorporated by reference herein.

### FIELD OF THE INVENTION

This invention is related to a method of contraception and/or hormone replacement therapy (“HRT”) that provides for the reduced level of estrogen in the each of the phases in the multiphasic estrogenic/progestogenic contraceptive and/or HRT regimen, as well as an overall reduced level of estrogen in the regimen without compromising contraceptive efficacy, cycle control, relief of menopausal symptoms or other desired benefits. The invention is also provides a multiphasic contraceptive and/or HRT kit that may be used to practice the aforementioned inventive method.

### BACKGROUND OF THE INVENTION

Contraceptive compositions contain both estrogenic and progestogenic compounds. The progestogenic component of the composition is primarily responsible for the contraceptive efficacy of the composition, and the estrogenic component is employed to reduce undesired side effects, such as breakthrough bleeding or spotting.

Earlier forms of estrogenic/progestogenic contraceptive compositions contained a relatively high level of estrogenic component. Over time estrogenic/progestogenic contraceptive compositions have been disclosed where the amount of estrogen of such compositions has been lowered without reducing contraceptive efficacy and/or increasing undesired side effects. U.S. Pat. No. 5,888,543, discloses progestogen/estrogen combinations in a monophasic regimen (fixed dose in the cycle) or as biphasic or triphasic regimens (varied dose over the cycle).

U.S. Pat. No. 4,962,098 discloses a triphasic method of contraception using a progestogen/estrogen combination in which the amount of estrogen is increased stepwise over the three phases. The first phase is 4-7 days, the second phase is 5-8 days and the third phase is 7-12 days. Preferably, the administration of the contraceptive compositions for the three phases will be 21 days followed by a 7 day placebo period. For all three phases the progestogen is 0.5 to 1.5 mg of norethindrone acetate, while about 10 to 30 mcg of ethinyl estradiol is used in the first phase, about 20 to 40 mcg of ethinyl estradiol is used in the second phase and 30 to 50 mcg of ethinyl estradiol is employed in the third phase. Other multiphasic contraceptive compositions have been described in U.S. Pat. Nos. 8,461,138 and 8,124,595.

The treatment of menopausal symptoms such as hot flashing, osteoporosis and other symptoms associated with hormone deficiency is also known. Typically, such treatment involves continuous, daily administration of at least one hormonal ingredient.

### SUMMARY OF THE INVENTION

The regimens disclosed herein make it possible, to have an effective contraceptive product as well as to obtain hormone replacement benefits. The invention provides a multiphasic method of contraception that makes it possible to reduce the amount of administered estrogen while still achieving desired contraceptive efficacy and controlling undesired side effects. In addition, this multiphasic method may be used as a HRT regimen that can provide relief from hot flashes, osteoporosis and other conditions associated with hormone deficiency.

In an embodiment, the method of contraception or hormone replacement therapy is a multiphasic regimen with varying doses of an estrogen without a placebo period. In accordance with this embodiment, a further daily dose of an estrogen is administered in place of a placebo period.

In an embodiment, the method of contraception or hormone replacement therapy is a multiphasic regimen with varying doses of an estrogen with a placebo period.

In an embodiment, the method of contraception or HRT comprises the steps of sequentially administering, to a female in need thereof: (a) a phase I composition comprising a progestogen in an amount equivalent to about 0.3 mg to about 1.5 mg norethindrone acetate and an estrogen in an amount equivalent to about 2 mcg to about 5 mcg of ethinyl estradiol for about 3 to about 7 days; (b) a phase II composition comprising a progestogen in an amount equivalent to about 0.3 mg to about 1.5 mg norethindrone acetate and an estrogen in an amount equivalent to about 4 mcg to less than about 10 mcg of ethinyl estradiol for about 5 to about 9 days; (c) a phase III composition comprising a progestogen in an amount equivalent to about 0.3 mg to about 1.5 mg norethindrone acetate and an estrogen in an amount equivalent to about 5 mcg to less than about 15 mcg of ethinyl estradiol for about 10 to about 14 days; and (d) a phase IV composition comprising a placebo or an estrogen in an amount equivalent to about 5 mcg to about 15 mcg of ethinyl estradiol and substantially free of norethindrone acetate for about 1 to about 4 days, wherein the amount of estrogen in the phase II composition is greater than the amount of estrogen in the phase I composition.

In an embodiment, the method of contraception or HRT may also comprise administering a phase V composition following the completion of the phase IV composition which was not a placebo, wherein the phase V composition is a placebo administered for about 1 day to about 4 days, and wherein the sequential administration of phase I, II, III, IV and V is repeated upon completion of the administration of the phase V composition.

In an embodiment, the method of contraception or HRT comprises administering to a female in need thereof in sequential phases I-IV: (a) a total amount of a progestogen in an amount equivalent to 10.5 mg or less of norethindrone acetate in equal daily doses and a total amount of an estrogen in an amount equivalent to 35 mcg or less of ethinyl estradiol in equal daily doses in the phase I for about 3 to about 7 days, provided that an amount equivalent to at least 0.3 mg of norethindrone acetate and an amount equivalent to at least 2 mcg of ethinyl estradiol are administered daily; (b) a total amount of a progestogen in an amount equivalent to 13.5 mg or less of norethindrone acetate in equal daily doses and a total amount of an estrogen in an amount equivalent to 81 mcg or less of ethinyl estradiol in equal daily doses in the phase II for about 5 to about 9 days, provided that an amount equivalent to at least 0.3 mg of norethindrone acetate and an amount equivalent to at least 4 mcg of ethinyl estradiol are administered daily; (c) a total amount of a progestogen in an amount equivalent to 21 mg or less of norethindrone acetate in equal daily doses and a total amount of an estrogen in an amount equivalent to 196 mcg or less of ethinyl estradiol in equal daily doses in the phase III for about 10 to about 14 days, provided that an amount equivalent to at least 0.3 mg of norethindrone acetate and an amount equivalent to at least 5 mcg of ethinyl estradiol are administered daily; and (d)(i) a placebo or (ii) a total amount of an estrogen in an amount equivalent to 56 mcg or less of ethinyl estradiol in equal daily doses, provided that an amount equivalent to at least 5 mcg of ethinyl estradiol is administered daily, in phase IV for about 1 to about 4 days, and wherein substantially no norethindrone acetate is administered in the phase IV.

In an embodiment, when estrogen is administered in the phase IV, the method further comprises a phase V comprising administering a placebo for about 1 to about 4 days, and wherein the sequence of phase I, II, III, IV and V is repeated upon completion of the phase V.

Yet another embodiment provides a multiphasic contraceptive and/or HRT kit comprising a package comprising daily dosages of: a phase I composition comprising a progestogen in an amount equivalent to about 0.3 mg to about 1.5 mg norethindrone acetate and an estrogen in an amount equivalent to about 0.5 to about 5 mcg of ethinyl estradiol; a phase II composition comprising a progestogen in an amount equivalent to about 0.3 to about 1.5 mg norethindrone acetate and an estrogen in an amount equivalent to about 2 to about 9 mcg of ethinyl estradiol; a phase III composition comprising a progestogen in an amount equivalent to about 0.3 to about 1.5 mg norethindrone acetate and an estrogen in an amount equivalent to about 5 to less than about 15 mcg of ethinyl estradiol; and a phase IV composition comprising an estrogen in an amount equivalent to about 5 to less than about 15 mcg of ethinyl estradiol or a placebo. When the phase IV composition was not a placebo, the kit can further comprise a phase V composition wherein the phase V composition is a placebo.

### DETAILED DESCRIPTION OF THE INVENTION

The regimens disclosed herein, in addition to delivering safe and effective contraceptive efficacy, can also be used for HRT so as to provide effective treatment or prevention of menopausal symptoms, such as hot flushes, osteoporosis and valvovaginal atrophy, as well other symptoms associated with hormone deficiency. As a result, there is no need to switch from a contraceptive regimen to a hormone replacement regimen that is traditionally made.

As females move from fertile (e.g., premenopause) to perimenopause to menopause, a desire for contraceptives to prevent pregnancy can also become accompanied by a need for hormone replacement therapy to address symptoms associated with menopause. And, because this transition is not necessarily readily apparent and, in fact, may not be the same for any individual female, elimination of the need to make a switch from an oral contraceptive to an HRT therapy can help avoid unwanted pregnancies and address the many symptoms of perimenopause and menopause, including hot flashes, night sweats, vaginal dryness, aches and pains, insomnia and cognitive changes (e.g. memory loss), which can sometimes be severe. The multiphasic regimens presented here however are designed to be effective at both contraception and addressing the symptoms of menopause and avoid the need to switch from an oral contraceptive to a HRT.

Another issue in the art was that multiphasic regimens still needed relatively high amounts of estrogen to be effective. Contrary to that conventional understanding, the multiphasic regimens disclosed herein can be effective even though they significantly reduce estrogen exposure over the complete cycle of phases compared to known multiphasic graduated contraceptive regimens, especially quadriphasic contraceptive regimens. For instance, the total amount of ethinyl estradiol administered in the cycle of 28 days may be reduced by 60% or more, 64% or more, 68% or more, or even as much as 71% or more, compared to conventional of 28-day multiphasic graduated contraception methods.

Specifically, it is possible to limit the amount of ethinyl estradiol (or its estrogenic equivalent) in at least the first and/or second phases of the regimen disclosed herein without compromising desired efficacy or cycle control. In particular, the multiphasic regimens disclosed herein make it possible to provide the desired contraceptive effect with significantly less estrogen (e.g., ethinyl estradiol) being administered in at least one phase compared to conventional multiphasic contraceptive regimens, especially multiphasic graduated contraceptive, such as quadriphasic regimens. In an embodiment, there is significantly less ethinyl estradiol administered in at least two phases (e.g., phases I and II) compared to conventional contraceptive multiphasic regimens. For example, the total amount of ethinyl estradiol administered over 7 days in phase II may be reduced by as much as 80% or more, or even as much as 83% or more, compared to that administered in accordance with conventional multiphasic graduated contraception methods.

Also, traditional HRT regimens, e.g., monophasic femhrt®, are not indicated for contraception and are not considered adequate to provide reliable contraception. These regimens typically involve continuous (no placebo period), daily hormone administration, and have been shown to have higher incidence of amenorrhea as reported in the femhrt® label. See, e.g., femhrt® Label (Revised 11/2017). Other examples of such HRT regimens are disclosed in U.S. Pat. No. 5,208,225. As mentioned above, the regimens disclosed herein can be used as HRT so as to provide effective treatment or prevention of menopausal symptoms.

Thus, contrary to conventional understanding that separate regimens are needed for contraceptive control and HRT, the present invention, especially for females older than 35, can regulate their menstrual cycles and minimize estrogen exposure, as well as avoid the need to switch from a contraceptive regimen to a hormone replacement regimen as the disclosed regimens can treat symptoms, such as hot flushes, osteoporosis and valvovaginal atrophy, as well other symptoms associated with hormone deficiency. Thus, there is no need to determine the precise time to make the switch from using a contraceptive regimen to HRT, which if done too soon with conventional therapies can lead to unwanted pregnancy.

The method as disclosed herein includes the sequential administration of phase I, II, III and IV compositions, and optionally a phase V composition, which can be used for contraception and/or HRT. When the method is a quadriphasic method, the sequential administration of phase I, II, III and IV compositions is repeated after the completion of the administration of the phase IV composition. When the method is a pentaphasic method, the sequential administration of phase I, II, III, IV and V compositions is repeated after the completion of the administration of the phase V composition.

The total administration time for the quadriphasic or pentaphasic method may range from a 20 to a 34 day period. In an embodiment, the total administration time for phase I compositions, phase II compositions, phase III compositions, phase IV composition(s), and optionally phase V composition(s), is about 22 days, or about 23 days, or about 24 days, or about 25 days or about 26 days, or about 27 days, or about 28 days, or about 29 days, or about 30 days, or about 31 days, or about 32 days or about 33 days, or about 34 days. In an embodiment, the total administration time for phase I, II, III, and IV compositions is 22 to 30 days. In an embodiment, the total administration time for phase I, II, III, IV and V compositions is 22 to 30 days. In an embodiment, the total administration time for phase I, II, III, and IV compositions is 28 days. In an embodiment, the total administration time for phase I, II, III, IV and V compositions is 28 days.

Estrogens which may be used in the present invention include, for example, ethinyl estradiol, 17β-estradiol, 17β-estradiol-3-acetate, mestranol, conjugated estrogens, USP and estrone or salts thereof. The amount of estrogen used is described herein as that which is “equivalent” in estrogenic potency to an amount of ethinyl estradiol. The equivalent estrogenic potency of an estrogen to ethinyl estradiol may be readily determined by one of ordinary skill in the art. It is contemplated that each phase could employ one or more different estrogens that deliver a potency equivalent to the recited amount of ethinyl estradiol. It is also contemplated that the estrogen used in one phase may be different than that used in another phase. In an embodiment, the estrogen for each phase is ethinyl estradiol.

Progestogens which may be used in the present invention include, for example, progesterone and its derivatives such as 17-hydroxy progesterone esters and 19-nor-17-hydroxy progesterone esters, 17-alpha-ethinyl testosterone, 17-alpha-ethinyl-19-nortestosterone (norethindrone) and derivatives thereof, norethindrone acetate, norgestrel, nogestamate, desogestrel and D-17-beta-acetoxy-17-beta-ethyl-17-alpha-ethinyl-gon-4-en-3-one oxime. Other exemplary progestogens include demegestone, drospirenone, dydrogesterone, gestodene, medrogestone, medroxy progesterone and esters thereof. The amount of progestogen used is described herein as that which is “equivalent” in progestogenic potency to an amount of norethindrone acetate. The equivalent progestogenic potency of a progestogen to norethindrone acetate may be readily determined by one of ordinary skill in the art. It is contemplated that each phase could employ one or more different progestogens that deliver a potency equivalent to the recited amount of norethindrone acetate. It is also contemplated that the progestogen used in one phase may be different than that used in another phase. In an embodiment, the progestogen for each of phase I, II and III is norethindrone acetate.

“Administration” as used herein refers to oral administration. That is, the methods/regimens disclosed herein pertain to oral administrations, and the phase I-V compositions are suitable for oral administration.

The designation “mcg” refers to micrograms and “mg” to milligrams.

The term “a female in need thereof” refers to a human female of child bearing age, a pen-menopausal female, and/or a menopausal female. The female in need thereof may be 35 years of age or younger or may be over the age of 35. The female in need thereof may weigh 180 pounds or less or may weigh over 180 pounds. The female in need thereof may have a body mass index (BMI) of less than 30 kg/m² or at least 30 kg/m². In an embodiment, the female in need thereof is older than 35 years old and weighs 180 pounds or less. In another embodiment, the female in need thereof is older than 35 years old and has a BMI of less than 30 kg/m².

In an embodiment, the method includes administering, in sequential steps, to a female in need thereof, the following compositions: (a) phase I composition for about 3 to about 7 days comprising a progestogen in an amount equivalent to about 0.3 mg to about 1.5 mg norethindrone acetate and an estrogen in an amount equivalent to about 2 mcg to about 5 mcg of ethinyl estradiol; (b) phase II composition for about 5 to about 9 days comprising a progestogen in an amount equivalent to about 0.3 mg to about 1.5 mg norethindrone acetate and an estrogen in an amount equivalent to about 4 mcg to less than about 10 mcg of ethinyl estradiol; (c) phase III composition comprising a progestogen in an amount equivalent to about 0.3 mg to about 1.5 mg norethindrone acetate and an estrogen in an amount equivalent to about 5 mcg to less than about 15 mcg of ethinyl estradiol for about 10 to about 14 days; (d) phase IV composition comprising a placebo or an estrogen in an amount equivalent to about 5 mcg to less than about 15 mcg of ethinyl estradiol and substantially free of progestogen (e.g., norethindrone acetate) for about 1 to about 4 days; and, optionally, (e) phase V composition comprising a placebo for about 1 to about 4 days administered if phase IV is not a placebo.

In an embodiment, the phase I, II and III compositions all contain a progestogen and increasing amounts estrogen, and the phase IV composition is substantially free of progestogen. In an embodiment, the amount of estrogen in the phase II composition is greater than the amount of estrogen in the phase I composition. In an embodiment, the amount of estrogen is the phase III composition is greater than the amount of estrogen in phase II and phase I compositions. In an embodiment, there is a different amount of estrogen in each of the phase I, II and III compositions. In an embodiment, the phase I composition and the phase II composition have an estrogen in an amount equivalent to less than 10 mcg of ethinyl estradiol. In an embodiment, the phase I, II, III and IV compositions have an estrogen in an amount equivalent to 10 mcg or less of ethinyl estradiol.

In an embodiment, the phase I composition comprises an estrogen in an amount equivalent to about 2 mcg to less than 5 mcg of ethinyl estradiol. In an embodiment, the phase I composition comprises an estrogen in an amount equivalent to about 2 mcg to about 4 mcg, or about 2.25 mcg to about 3.5 mcg, or about 2.25 mcg to about 3 mcg of ethinyl estradiol. In an embodiment, the phase I composition comprises an estrogen in an amount equivalent about 2 mcg, or about 2.25 mcg, or about 2.5 mcg, or about 2.75 mcg, or about 3 mcg, or about 3.5 mcg, or about 4 mcg, or about 4.5 mcg, or about 5 mcg of ethinyl estradiol and any amount in between.

In an embodiment, the phase I composition comprises a progestogen in an amount equivalent to about 0.4 mg to about 1.3 mg, or about 0.4 mg to about 1.1 mg, or about 0.5 mg to about 1 mg of norethindrone acetate. In an embodiment, the phase I composition comprises a progestogen in an amount equivalent to about 0.4 mg, or about 0.5 mg, or about 0.6 mg, or about 0.7 mg, or about 0.8 mg, or about 0.9 mg, or about 1 mg, or about 1.1 mg, or about 1.2 mg, or about 1.3 mg of norethindrone acetate and any amount in between.

In an embodiment, the phase I composition comprises about 2.5 mcg of ethinyl estradiol and about 0.5 mg of norethindrone acetate or about 1 mg of norethindrone acetate.

In an embodiment, the total amount of an estrogen administered in phase I is an amount equivalent to 35 mcg or less of ethinyl estradiol in equal daily doses. In an embodiment, the total amount of an estrogen administered in phase I is an amount equivalent to about 8 mcg to about 25 mcg, or about 8.5 mcg to about 20 mcg, or about 9 mcg to about 15 mcg, of ethinyl estradiol in equal daily doses. In an embodiment, the total amount of an estrogen administered in phase I is an amount equivalent to about 9 mcg, or about 9.5 mcg, or about 10 mcg, or about 10.5 mcg, or about 11 mcg, or about 11.5 mcg, or about 12 mcg, or about 12.5 mcg, about 13 mcg, or about 13.5 mcg, or about 14 mcg, or about 14.5 mcg, or about 15 mcg, or about 15.5 mcg, or about 16 mcg, and any amount in between, of ethinyl estradiol in equal daily doses. The minimum of estrogen administered daily in phase I is as described in this disclosure in connection with the phase I compositions.

In an embodiment, the total amount of a progestogen administered in phase I is an amount equivalent to 10.5 mg or less of norethindrone acetate in equal daily doses. In an embodiment, the total amount of progestogen administered in phase I is an amount equivalent to about 1 mg to about 8 mg, or about 1.5 mg to about 7 mg, or about 1.75 mg to about 6 mg, of norethindrone acetate in equal daily doses. In an embodiment, the total amount of progestogen administered in phase I is an amount equivalent to about 2 mg, or about 2.5 mg, or about 3 mg, or about 3.5 mg, or about 4 mg, or about 4.5 mg, or about 5 mg, or about 5.5 mg or about 6 mg, or about 6.5 mg, or about 7 mg, or about 7.5 mg, or about 8 mg, and any amount in between, of norethindrone acetate in equal daily doses. The minimum amount of progestogen administered daily in phase I is as described in this disclosure in connection with the phase I compositions.

In an embodiment, the total amount of ethinyl estradiol administered in phase I is about 10 mcg or about 12.5 mcg, in equal daily doses, and the total amount of norethindrone acetate administered in phase I is about 2 mg or about 5 mg, in equal daily doses. The minimum amounts of ethinyl estradiol and norethindrone acetate administered daily are as described in this disclosure in connection with the phase I compositions.

In an embodiment, the phase II composition comprises an estrogen in an amount equivalent to about 4 mcg to about 9 mcg, or about 4 mcg to about 8 mcg, or about 4 mcg to about 7 mcg, or about 4 to about 6 mcg of ethinyl estradiol. In an embodiment, the phase II composition comprises an estrogen in an amount equivalent to about 4 mcg, or about 4.5 mcg, or about 5 mcg, or about 5.5 mcg, or about 6 mcg, or about 6.5 mcg, or about 7 mcg, or about 7.5 mcg, or about 8 mcg, or about 8.5 mcg, or about 9 mcg of ethinyl estradiol and any amount in between.

In an embodiment, the phase II composition comprises a progestogen in an amount equivalent to about 0.4 mg to about 1.3 mg, or about 0.4 mg to about 1.1 mg, or about 0.5 mg to about 1 mg of norethindrone acetate. In an embodiment, the phase II composition comprises a progestogen in an amount equivalent to about 0.4 mg, or about 0.5 mg, or about 0.6 mg, or about 0.7 mg, or about 0.8 mg, or about 0.9 mg, or about 1 mg, or about 1.1 mg, or about 1.2 mg, or about 1.3 mg of norethindrone acetate and any amount in between.

In an embodiment, the phase II composition comprises about 5 mcg of ethinyl estradiol and about 1 mg of norethindrone acetate.

In an embodiment, the total amount of an estrogen administered in phase II is an amount equivalent to 81 mcg or less of ethinyl estradiol in equal daily doses. In an embodiment, the total amount of an estrogen administered in phase II is an amount equivalent to about 20 mcg to about 60 mcg, or about 25 mcg to about 55 mcg, or about 28 mcg to about 50 mcg, or about 30 mcg to about 45 mcg, of ethinyl estradiol in equal daily doses. In an embodiment, the total amount of estrogen administered in phase II is an amount equivalent to about 25 mcg, or about 30 mcg, or about 35 mcg, or about 40 mcg, or about 45 mcg, or about 50 mcg, or about 55 mcg, or about 60 mcg and any amount in between, of ethinyl estradiol in equal daily doses. The minimum of estrogen administered daily in phase II is as described in this disclosure in connection with the phase II compositions.

In an embodiment, the total amount of a progestogen in phase II is an amount equivalent to 13.5 mg or less of norethindrone acetate in equal daily doses. In an embodiment, the total amount of progestogen administered in phase II is an amount equivalent to about 4 mg to about 13.5 mg, or about 5 mg to about 12 mg or about 6 mg to about 10 mg, of norethindrone acetate in equal daily doses. In an embodiment, the total amount of progestogen administered in phase II is an amount equivalent to about 4 mg, or about 5 mg, or about 5.5 mg, or about 6 mg, or about 6.5 mg, or about 7 mg, or about 7.5 mg, or about 8 mg, or about 8.5 mg, or about 9 mg, or about 9.5 mg, or about 10 mg, or about 10.5 mg, or about 11 mg, or about 11.5 mg, or about 12 mg and any amount in between, of norethindrone acetate in equal daily doses. The minimum amount of progestogen administered daily in phase II is as described in this disclosure in connection with the phase II compositions.

In an embodiment, the total amount of ethinyl estradiol administered in phase II is about 35 mcg or about 40 mcg, in equal daily doses, and the total amount of norethindrone acetate administered in phase I is about 7 mg or about 8 mg, in equal daily doses. The minimum amounts of ethinyl estradiol and norethindrone acetate administered daily are as described in this disclosure in connection with the phase II compositions.

In an embodiment, the phase III composition comprises an estrogen in an amount equivalent to about 6 mcg to about 14 mcg, or about 7 mcg to about 14 mcg, or about 8 mcg to about 13 mcg, or about 9 mcg to about 11 mcg of ethinyl estradiol. In an embodiment, the phase III composition comprises an estrogen in an amount equivalent to about 6 mcg, or about 6.5 mcg, or about 7 mcg, or about 7.5 mcg, or about 8 mcg, or about 8.5 mcg, or about 9 mcg, or about 9.5 mcg, or about 10 mcg, or about 10.5 mcg, or about 11 mcg, or about 11.5 mcg, or about 12 mcg, or about 12.5 mcg, or about 13 mcg, or about 13.5 mcg, or about 14 mcg of ethinyl estradiol and any amount in between.

In an embodiment, the phase III composition comprises a progestogen in an amount equivalent to about 0.4 mg to about 1.3 mg, or about 0.4 mg to about 1.1 mg, or about 0.5 mg to about 1 mg of norethindrone acetate. In an embodiment, the phase III composition comprises a progestogen in an amount equivalent to about 0.4 mg, or about 0.5 mg, or about 0.6 mg, or about 0.7 mg, or about 0.8 mg, or about 0.9 mg, or about 1 mg, or about 1.1 mg, or about 1.2 mg, or about 1.3 mg of norethindrone acetate and any amount in between.

In an embodiment, the phase III composition comprises about 10 mcg of ethinyl estradiol and about 1 mg of norethindrone acetate.

In an embodiment, the total amount of an estrogen in phase III is an amount equivalent to 196 mcg or less of ethinyl estradiol in equal daily doses. In an embodiment, the total amount of an estrogen administered in phase III is an amount equivalent to about 70 mcg to about 195 mcg, or about 80 mcg to about 180 mcg, or about 90 mcg to about 160 mcg, or about 100 mcg to about 150 mcg, or about 110 mcg to about 130 mcg, of ethinyl estradiol in equal daily doses. In an embodiment, the total amount of an estrogen administered in phase III is an amount equivalent to about 80 mcg, or about 90 mcg, or about 100 mcg, or about 110 mcg, or about 120 mcg or about 130 mcg or about 140 mcg, or about 150 mcg, or about 160 mcg, or about 170 mcg, or about 180 mcg, or about 190 mcg, and any amount in between, of ethinyl estradiol in equal daily doses. The minimum amount of estrogen administered daily in phase III is as described in this disclosure in connection with the phase III compositions.

In an embodiment, the total amount of a progestogen in phase III is an amount equivalent to 21 mg or less of norethindrone acetate in equal daily doses. In an embodiment, the total amount of a progestogen administered in phase III is an amount equivalent to about 5 mg to about 20 mg, or about 6 mg to about 15 mg, or about 10 mg to about 14 mg, of norethindrone acetate in equal daily doses. In an embodiment, the total amount of a progestogen administered in phase III is an amount equivalent to about 6 mg, or about 6.5 mg, or about 7 mg, or about 7.5 mg, or about 8 mg, or about 8.5 mg, or about 9 mg, or about 9.5 mg, or about 10 mg, or about 10.5 mg, or about 11 mg, or about 11.5 mg, or about 12 mg, or about 12.5 mg, or about 13 mg, or about 13.5 mg, or about 14 mg, or about 14.5 mg, or about 15 mg, of norethindrone acetate and any amount in between, in equal daily doses. The minimum amount of progestogen administered daily in phase III is as described in this disclosure in connection with the phase III compositions.

In an embodiment, the total amount of ethinyl estradiol administered in phase III is about 120 mcg, in equal daily doses, and the total amount of norethindrone acetate administered in phase III is about 12 mg, in equal daily doses. The minimum amounts of ethinyl estradiol and norethindrone acetate administered daily are as described in this disclosure in connection with the phase III compositions.

In an embodiment, the phase IV composition is an unopposed estrogen composition. In an embodiment, the phase IV composition comprises an estrogen in an amount equivalent to about 5 mcg to about 14 mcg, or about 5 mcg to about 10 mcg, or about 7 mcg to about 14 mcg, or about 8 mcg to about 13 mcg, or about 9 mcg to about 11 mcg of ethinyl estradiol, and is substantially free of progestogen (e.g., norethindrone acetate). In an embodiment, the phase IV composition comprises an estrogen in an amount equivalent to about 5 mcg, or about 5.5 mcg, or about 6 mcg, or about 6.5 mcg, or about 7 mcg, or about 7.5 mcg, or about 8 mcg, or about 8.5 mcg, or about 9 mcg, or about 9.5 mcg, or about 10 mcg, or about 10.5 mcg, or about 11 mcg, or about 11.5 mcg, or about 12 mcg, or about 12.5 mcg, or about 13 mcg, or about 13.5 mcg, or about 14 mcg of ethinyl estradiol and any amount in between, and is substantially free of progestogen (e.g., norethindrone acetate).

In an embodiment, the phase IV composition comprises about 5 mcg or about 10 mcg of ethinyl estradiol and is substantially free of norethindrone acetate.

In an embodiment, total amount of an estrogen in phase IV is an amount equivalent to 56 mcg or less of ethinyl estradiol in equal daily doses. In an embodiment, the total amount of an estrogen in phase IV is an amount equivalent to about 5 mcg to about 50 mcg, or about 10 mcg to about 40 mcg, or about 15 to about 25 mcg, of ethinyl estradiol in equal daily doses, and is substantially free of progestogen (e.g., norethindrone acetate). In an embodiment, the total amount of an estrogen in phase IV is an amount equivalent to about 10 mcg, or about 15 mcg, or about 20 mcg, or about 25 mcg, or about 30 mcg, or about 35 mcg, or about 40 mcg, or about 45 mcg, or about 50 mcg, and any amount in between, of ethinyl estradiol in equal daily doses. The minimum amount of estrogen administered daily in phase IV is as described in this disclosure in connection with the phase IV compositions.

In an embodiment, the phase IV composition is a placebo.

In an embodiment, the phase I composition is administered for about 5 days, and/or phase II composition is administered for about 7 days, and/or phase III composition is administered for about 12 days, and/or phase IV composition is administered for about 4 days.

In an embodiment, the phase I composition is administered for about 5 days, and/or phase II composition is administered for about 7 days, and/or phase III composition is administered for about 12 days, and/or phase IV composition is administered for about 2 days, and/or phase V is administered for about 2 days. In an embodiment, the phase I composition is administered for about 5 days, and/or phase II composition is administered for about 7 days, and/or phase III composition is administered for about 12 days, and/or phase IV composition is administered for about 3 days, and/or phase V is administered for about 1 day. In an embodiment, the phase I composition is administered for about 5 days, and/or phase II composition is administered for about 7 days, and/or phase III composition is administered for about 12 days, and/or phase IV composition is administered for about 1 day, and/or phase V is administered for about 3 days.

In an embodiment, the phase I composition is administered for about 4 days, and/or phase II composition is administered for about 8 days, and/or phase III composition is administered for about 12 days, and/or phase IV composition is administered for about 2 days, and/or phase V is administered for about 2 days. In an embodiment, the phase I composition is administered for about 4 days, and/or phase II composition is administered for about 8 days, and/or phase III composition is administered for about 12 days, and/or phase IV composition is administered for about 3 days, and/or phase V is administered for about 1 day. In an embodiment, the phase I composition is administered for about 4 days, and/or phase II composition is administered for about 8 days, and/or phase III composition is administered for about 12 days, and/or phase IV composition is administered for about 1 day, and/or phase V is administered for about 3 days.

In an embodiment, the phase I composition comprises about 1 mg of norethindrone acetate and about 2.5 mcg of ethinyl estradiol, the phase II composition comprises about 1 mg of norethindrone acetate and about 5 mcg of ethinyl estradiol, the phase III composition comprises about 1 mg of norethindrone acetate and about 10 mcg of ethinyl estradiol, and the phase IV composition comprises about 10 mcg of ethinyl estradiol and is substantially free of norethindrone acetate or is a placebo.

In an embodiment, the phase I composition comprises about 1 mg of norethindrone acetate and about 2.5 mcg of ethinyl estradiol, the phase II composition comprises about 1 mg of norethindrone acetate and about 5 mcg of ethinyl estradiol, the phase III composition comprises about 1 mg of norethindrone acetate and about 10 mcg of ethinyl estradiol, and the phase IV composition comprises about 5 mcg of ethinyl estradiol and is substantially free of norethindrone acetate or is a placebo.

In an embodiment, the phase I composition comprises about 0.5 mg of norethindrone acetate and about 2.5 mcg of ethinyl estradiol, the phase II composition comprises about 1 mg of norethindrone acetate and about 5 mcg of ethinyl estradiol, the phase III composition comprises about 1 mg of norethindrone acetate and about 10 mcg of ethinyl estradiol, and the phase IV composition comprises about 10 mcg of ethinyl estradiol and is substantially free of norethindrone acetate or is a placebo.

In an embodiment, the phase I composition comprises about 0.5 mg of norethindrone acetate and about 2.5 mcg of ethinyl estradiol, the phase II composition comprises about 1 mg of norethindrone acetate and about 5 mcg of ethinyl estradiol, the phase III composition comprises about 1 mg of norethindrone acetate and about 10 mcg of ethinyl estradiol, and the phase IV composition comprises about 5 mcg of ethinyl estradiol and is substantially free of norethindrone acetate or is a placebo.

In an embodiment, the phase I composition comprises about 0.5 mg of norethindrone acetate and about 2.5 mcg of ethinyl estradiol, the phase II composition comprises about 1 mg of norethindrone acetate and about 5 mcg of ethinyl estradiol, the phase III composition comprises about 1 mg of norethindrone acetate and about 10 mcg of ethinyl estradiol, the phase IV composition comprises about 10 mcg of ethinyl estradiol and is substantially free of norethindrone acetate, and the phase V composition is a placebo.

In an embodiment, the phase I composition comprises about 0.5 mg of norethindrone acetate and about 2.5 mcg of ethinyl estradiol, the phase II composition comprises about 1 mg of norethindrone acetate and about 5 mcg of ethinyl estradiol, the phase III composition comprises about 1 mg of norethindrone acetate and about 10 mcg of ethinyl estradiol, the phase IV composition comprises about 5 mcg of ethinyl estradiol and is substantially free of norethindrone acetate, and the phase V composition is a placebo.

In an embodiment, a multiphasic contraceptive and/or hormone replacement therapy is a kit comprising a package containing daily dosages of phase I, II, III, IV compositions, and optionally, phase V composition, as described herein. The compositions may be placed, for example, in a blister pack. The compositions for each phase may be provided in different colors and/or the packaging can be labeled to indicate the order of administration.

The compositions used in this invention are administered using a suitable daily dosage form. Tablets, pills, capsules and caplets are exemplary dosage forms.

Suitable carriers with which the compositions can be administered include lactose, starch, cellulose derivatives and the like used in suitable amounts. Mixtures of carriers, e.g. lactose, microcrystalline cellulose and starch, are operable.

Reasonable variations, such as those which would occur to a skilled artisan, can be made herein without departing from the scope of the invention.

While the methods/regimens as disclosed herein are practiced by administration of the compositions in a numeric sequence with the phase I composition being used first, the phase II composition being used second, phase III composition being used third, the phase IV composition being used fourth, and, optionally, the phase V composition being used fifth, if packaging and/or other requirements dictate, the method and kit described herein can be employed as part of a larger scheme for contraception, hormone replacement therapy or treatment of gynecological disorders. Even though the sequence in which the compositions are administered is important to their operation, it should be kept in mind that variations in timing and dosage can be tolerated when medical considerations so dictate.

### EXAMPLES

Specific embodiments will now be demonstrated by reference to the following examples. It should be understood that these examples are disclosed solely by way of illustrating the invention and should not be taken in any way to limit the scope of the present invention.

### Example 1

The compositions employed in accordance with the invention in phases I through V will have the administration times and drug contents set forth in the following table.

TABLE 1

| | | Norethindrone | Ethinyl |
|---|---|---|---|
| | | acetate | Estradiol |
| Phase | Days | (NA) (mg) | (EE) (mcg) |
| I | 5 | 1.0 | 2.5 |
| II | 7 | 1.0 | 5 |
| III | 12 | 1.0 | 10 |
| IV | 2 | n/a | 10 |
| V | 2 | n/a (placebo) | n/a (placebo) |

The norethindrone acetate (NA) and ethinyl estradiol (EE) are well known and readily available.

### Example 2

The compositions employed in accordance with the invention in phases I through IV will have the administration times and drug contents set forth in the following table.

TABLE 2

| | | Norethindrone | Ethinyl |
|---|---|---|---|
| | | acetate | Estradiol |
| Phase | Days | (NA) (mg) | (EE) (mcg) |
| I | 5 | 1.0 | 2.5 |
| II | 7 | 1.0 | 5 |
| III | 12 | 1.0 | 10 |
| IV | 4 | n/a (placebo) | n/a (placebo) |

The norethindrone acetate (NA) and ethinyl estradiol (EE) are well known and readily available.

### Example 3

The compositions employed in accordance with the invention in phases I through IV will have the administration times and drug contents set forth in the following table.

TABLE 3

| | | Norethindrone | Ethinyl |
|---|---|---|---|
| | | acetate | Estradiol |
| Phase | Days | (NA) (mg) | (EE) (mcg) |
| I | 4 | 1.0 | 2.5 |
| II | 8 | 1.0 | 5 |
| III | 12 | 1.0 | 10 |
| IV | 4 | n/a (placebo) | n/a (placebo) |

The norethindrone acetate (NA) and ethinyl estradiol (EE) are well known and readily available.

### Example 4

The compositions employed in accordance with the invention in phases I through IV will have the administration times and drug contents set forth in the following table.

TABLE 4

| | | Norethindrone | Ethinyl |
|---|---|---|---|
| | | acetate | Estradiol |
| Phase | Days | (NA) (mg) | (EE) (mcg) |
| I | 5 | 1.0 | 2.5 |
| II | 7 | 1.0 | 5 |
| III | 12 | 1.0 | 10 |
| IV | 4 | n/a | 10 |

The norethindrone acetate (NA) and ethinyl estradiol (EE) are well known and readily available.

### Example 5

The compositions employed in accordance with the invention in phases I through IV will have the administration times and drug contents set forth in the following table.

TABLE 5

| | | Norethindrone | Ethinyl |
|---|---|---|---|
| | | acetate | Estradiol |
| Phase | Days | (NA) (mg) | (EE) (mcg) |
| I | 4 | 1.0 | 2.5 |
| II | 8 | 1.0 | 5 |
| III | 12 | 1.0 | 10 |
| IV | 4 | n/a | 10 |

The norethindrone acetate (NA) and ethinyl estradiol (EE) are well known and readily available.

### Example 6

The compositions employed in accordance with the invention in phases I through V will have the administration times and drug contents set forth in the following table.

TABLE 6

| | | Norethindrone | Ethinyl |
|---|---|---|---|
| | | acetate | Estradiol |
| Phase | Days | (NA) (mg) | (EE) (mcg) |
| I | 4 | 0.5 | 2.5 |
| II | 8 | 1.0 | 5 |
| III | 12 | 1.0 | 10 |
| IV | 2 | n/a | 10 |
| V | 2 | n/a (placebo) | n/a (placebo) |

The norethindrone acetate (NA) and ethinyl estradiol (EE) are well known and readily available.

### Example 7

The compositions employed in accordance with the invention in phases I through V will have the administration times and drug contents set forth in the following table.

TABLE 7

| | | Norethindrone | Ethinyl |
|---|---|---|---|
| | | acetate | Estradiol |
| Phase | Days | (NA) (mg) | (EE) (mcg) |
| I | 5 | 0.5 | 2.5 |
| II | 7 | 1.0 | 5 |
| III | 12 | 1.0 | 10 |
| IV | 2 | n/a | 10 |
| V | 2 | n/a (placebo) | n/a (placebo) |

The norethindrone acetate (NA) and ethinyl estradiol (EE) are well known and readily available.

### Example 8

The compositions employed in accordance with the invention in phases I through V will have the administration times and drug contents set forth in the following table.

TABLE 8

| | | Norethindrone | Ethinyl |
|---|---|---|---|
| | | acetate | Estradiol |
| Phase | Days | (NA) (mg) | (EE) (mcg) |
| I | 5 | 1.0 | 2.5 |
| II | 7 | 1.0 | 5 |
| III | 12 | 1.0 | 10 |
| IV | 2 | n/a | 5 |
| V | 2 | n/a (placebo) | n/a (placebo) |

The norethindrone acetate (NA) and ethinyl estradiol (EE) are well known and readily available.

### Example 9

The compositions employed in accordance with the invention in phases I through V will have the administration times and drug contents set forth in the following table.

TABLE 9

| | | Norethindrone | Ethinyl |
|---|---|---|---|
| | | acetate | Estradiol |
| Phase | Days | (NA) (mg) | (EE) (mcg) |
| I | 4 | 0.5 | 2.5 |
| II | 8 | 1.0 | 5 |
| III | 12 | 1.0 | 10 |
| IV | 2 | n/a | 5 |
| V | 2 | n/a (placebo) | n/a (placebo) |

The norethindrone acetate (NA) and ethinyl estradiol (EE) are well known and readily available.

### Example 10

The compositions employed in accordance with the invention in phases I through V will have the administration times and drug contents set forth in the following table.

TABLE 10

| | | Norethindrone | Ethinyl |
|---|---|---|---|
| | | acetate | Estradiol |
| Phase | Days | (NA) (mg) | (EE) (mcg) |
| I | 5 | 1.0 | 2.5 |
| II | 7 | 1.0 | 5 |
| III | 12 | 1.0 | 10 |
| IV | 3 | n/a | 10 |
| V | 1 | n/a (placebo) | n/a (placebo) |

The norethindrone acetate (NA) and ethinyl estradiol (EE) are well known and readily available.

### Example 11

The compositions employed in accordance with the invention in phases I through V will have the administration times and drug contents set forth in the following table.

TABLE 11

| | | Norethindrone | Ethinyl |
|---|---|---|---|
| | | acetate | Estradiol |
| Phase | Days | (NA) (mg) | (EE) (mcg) |
| I | 4 | 0.5 | 2.5 |
| II | 8 | 1.0 | 5 |
| III | 12 | 1.0 | 10 |
| IV | 3 | n/a | 10 |
| V | 1 | n/a (placebo) | n/a (placebo) |

The norethindrone acetate (NA) and ethinyl estradiol (EE) are well known and readily available.

### Example 12

The compositions employed in accordance with the invention in phases I through V will have the administration times and drug contents set forth in the following table.

TABLE 12

| | | Norethindrone | Ethinyl |
|---|---|---|---|
| | | acetate | Estradiol |
| Phase | Days | (NA) (mg) | (EE) (mcg) |
| I | 5 | 1.0 | 2.5 |
| II | 7 | 1.0 | 5 |
| III | 12 | 1.0 | 10 |
| IV | 3 | n/a | 5 |
| V | 1 | n/a (placebo) | n/a (placebo) |

The norethindrone acetate (NA) and ethinyl estradiol (EE) are well known and readily available.

### Example 13

The compositions employed in accordance with the invention in phases I through V will have the administration times and drug contents set forth in the following table.

TABLE 13

| | | Norethindrone | Ethinyl |
|---|---|---|---|
| | | acetate | Estradiol |
| Phase | Days | (NA) (mg) | (EE) (mcg) |
| I | 4 | 0.5 | 2.5 |
| II | 8 | 1.0 | 5 |
| III | 12 | 1.0 | 10 |
| IV | 3 | n/a | 5 |
| V | 1 | n/a (placebo) | n/a (placebo) |

The norethindrone acetate (NA) and ethinyl estradiol (EE) are well known and readily available.

It should be noted that the table is presented for illustrative purposes only. The substitution of functionally equivalent amounts and kinds of reagent(s) in these schemes is contemplated.

1. A method of contraception or hormone replacement therapy (“HRT”) comprising the steps of sequentially administering daily to a female in need thereof:
a. a phase I composition comprising a progestogen in an amount equivalent to about 0.3 mg to about 1.5 mg norethindrone acetate and an estrogen in an amount equivalent to about 2 mcg to about 5 mcg of ethinyl estradiol for about 3 to about 7 days;
b. a phase II composition comprising a progestogen in an amount equivalent to about 0.3 mg to about 1.5 mg norethindrone acetate and an estrogen in an amount equivalent to about 4 mcg to less than about 10 mcg of ethinyl estradiol for about 5 to about 9 days;
c. a phase III composition comprising a progestogen in an amount equivalent to about 0.3 mg to about 1.5 mg norethindrone acetate and an estrogen in an amount equivalent to about 5 mcg to less than about 15 mcg of ethinyl estradiol for about 10 to about 14 days; and
d. a phase IV composition comprising a placebo or an estrogen in an amount equivalent to about 5 mcg to less than about 15 mcg of ethinyl estradiol and substantially free of norethindrone acetate for about 1 to about 4 days
wherein the amount of estrogen in the phase II composition is greater than the amount of estrogen in the phase I composition.

2. The method of claim 1, wherein sequential administration of the phase I, II, III and IV compositions is repeated upon completion of the administration of the phase IV composition.

3. The method of claim 1, further comprising administering a phase V composition following completion of the phase IV composition which was not a placebo, wherein the phase V composition is a placebo administrated for about 1 to about 4 days, and wherein the sequential administrations of phase I, II, III, IV and V compositions is repeated upon completion of the administration of the phase V composition.

4. The method of claim 1, wherein a total administration time for phase I compositions, phase II compositions, phase III compositions, and phase IV composition(s).

5. The method of claim 1, wherein the phase I composition is administered for about 5 days, and the phase II composition is administered for about 7 days, and the phase III composition is administered for about 12 days, and phase IV composition is administered for about 4 days.

6. The method of claim 1, wherein the phase I composition is administered for about 4 days, and the phase II composition is administered for about 8 days, and the phase III composition is administered for about 12 days, and the phase IV composition is administered for about 4 days.

7. The method of claim 3, wherein the phase I composition is administered for about 5 days, and the phase II composition is administered for about 7 days, and the phase III composition is administered for about 12 days, and the phase IV composition is administered for about 2 days, and the phase V composition is administered for about 2 days.

8. The method of claim 3, wherein the phase I composition is administered for about 5 days, and the phase II composition is administered for about 7 days, and the phase III composition is administered for about 12 days, and the phase IV composition is administered for about 3 days, and the phase V composition is administered for about 1 day.

9. The method of claim 3, wherein the phase I composition is administered for about 4 days, and the phase II composition is administered for about 8 days, and the phase III composition is administered for about 12 days, and the phase IV composition is administered for about 2 days, and the phase V composition is administered for about 2 days.

10. The method of claim 3, wherein the phase I composition is administered for about 4 days, and the phase II composition is administered for about 8 days, and the phase III composition is administered for about 12 days, and the phase IV composition is administered for about 3 days, and the phase V composition is administered for about 1 day.

11. The method of claim 1, wherein the progestogen in each composition is norethindrone acetate.

12. The method of claim 1, wherein the estrogen is each composition is ethinyl estradiol.

13. (canceled)

14. The method of claim 1, wherein the phase I composition comprises about 2.5 mcg of ethinyl estradiol and about 0.5 mg to about 1 mg of norethindrone acetate.

15. (canceled)

16. The method of claim 1, wherein the phase II composition comprises about 5 mcg of ethinyl estradiol and about 1 mg of norethindrone acetate.

17. (canceled)

18. The method of claim 1, wherein the phase III composition comprises about 10 mcg of ethinyl estradiol and about 1 mg of norethindrone acetate.

19. (canceled)

20. The method of claim 1, wherein the phase IV composition comprises about 5 mcg to about 10 mcg of ethinyl estradiol and is substantially free of norethindrone acetate.

21. The method of claim 1, wherein the phase IV composition is a placebo.

22.-31. (canceled)

32. A method of contraception or hormone replacement therapy (“HRT”) comprising administering to a female in need thereof in sequential phases I-IV:
a. a total amount of a progestogen in an amount equivalent to 10.5 mg or less of norethindrone acetate in equal daily doses and a total amount of an estrogen in an amount equivalent to 35 mcg or less of ethinyl estradiol in equal daily doses in phase I for about 3 to about 7 days, provided that an amount equivalent to at least 0.3 mg of norethindrone acetate and an amount equivalent to at least 2 mcg of ethinyl estradiol are administered daily;
b. a total amount of a progestogen in an amount equivalent to 13.5 mg or less of norethindrone acetate in equal daily doses and a total amount of an estrogen in an amount equivalent to 81 mcg or less of ethinyl estradiol in equal daily doses in phase II for about 5 to about 9 days, provided that an amount equivalent to at least 0.3 mg of norethindrone acetate and an amount equivalent to at least 4 mcg of ethinyl estradiol are administered daily;
c. a total amount of a progestogen in an amount equivalent to 21 mg or less of norethindrone acetate in equal daily doses and a total amount of an estrogen in an amount equivalent to 196 mcg or less of ethinyl estradiol in equal daily doses in phase III for about 10 to about 14 days, provided that an amount equivalent to at least 0.3 mg of norethindrone acetate and an amount equivalent to at least 5 mcg of ethinyl estradiol are administered daily; and
d. (i) a placebo; or
(ii) a total amount of an estrogen in an amount equivalent to 56 mcg or less of ethinyl estradiol in equal daily doses, provided that an amount equivalent to at least 5 mcg of ethinyl estradiol is administered daily,
in phase IV for about 1 to about 4 days, and wherein substantially no norethindrone acetate is administered in the phase IV.

33. The method of claim 32, wherein upon completion of the phase IV, the phases I, II, III and IV are repeated sequentially.

34. The method of claim 32, wherein when estrogen is administered in the phase IV, the method further comprises a phase V comprising administering a placebo for about 1 to about 4 days, and wherein the sequence of phase I, II, III, IV and V is repeated upon completion of the phase V.

35. The method of claim 32, wherein a total administration time for the phase I, the phase II, the phase III, and the phase IV.

36.-41. (canceled)

42. The method of claim 32, wherein the total amount of estrogen in the phase I, in the amount equivalent to ethinyl estradiol, is about 8 mcg to about 25 mcg, and the total amount of progestogen in phase I, in the amount equivalent to norethindrone acetate, is about 1 mg to about 8 mg.

43.-45. (canceled)

46. The method of claim 32, wherein the total amount of estrogen in the phase II, in the amount equivalent to ethinyl estradiol, is about 20 mcg to about 60 mcg, and the total amount of progestogen in the phase I, in the amount equivalent to norethindrone acetate, is about 4 mg to about 13.5 mg.

47.-49. (canceled)

50. The method of claim 32, wherein the total amount of estrogen in the phase III, in the amount equivalent to ethinyl estradiol, is about 70 mcg to about 195 mcg and the total amount of progestogen in phase III, in the amount equivalent to norethindrone acetate, is about 5 mg to about 20 mg.

51.-52.

53. The method of claim 32, wherein the total amount of estrogen in the phase IV, in the amount equivalent to ethinyl estradiol, is about 5 mcg to about 50 mcg, and wherein the phase IV is substantially free of progestogen.

54. (canceled)

55. The method of claim 32, wherein the progestogen in any of the phases I-III is norethindrone acetate.

56. The method of claim 32, wherein the estrogen in any of the phases I-IV is ethinyl estradiol.

57. A multiphase contraceptive and/or HRT kit comprising a package comprising daily dosages of:
a. a phase I composition comprising a progestogen in an amount equivalent to about 0.3 mg to about 1.5 mg norethindrone acetate and an estrogen in an amount equivalent to about 0.5 to about 5 mcg of ethinyl estradiol;
b. a phase II composition comprising a progestogen in an amount equivalent to about 0.3 to about 1.5 mg norethindrone acetate and an estrogen in an amount equivalent to about 2 to about 9 mcg of ethinyl estradiol;
c. a phase III composition comprising a progestogen in an amount equivalent to about 0.3 to about 1.5 mg norethindrone acetate and an estrogen in an amount equivalent to about 5 to less than about 15 mcg of ethinyl estradiol; and
d. a phase IV composition comprising an estrogen in an amount equivalent to about 5 to less than about 15 mcg of ethinyl estradiol or a placebo.

58. The kit of claim 57, further comprising a phase V composition, wherein the phase V composition is a placebo.

59.-86. (canceled)
